(19) United States
(12) Patent Application Publication
(10) Pub. No.: US 2008/0203258 A1
(43) Pub. Date: Aug. 28, 2008

(54) Apparatus for holding surgical instruments

(76) Inventor: Alexander Koenigstein, New York, NY (US)

Correspondence Address: EPSTEIN DRANGEL BAZERMAN & JAMES, LLP, 60 EAST 42ND STREET, SUITE 820, NEW YORK, NY, 10165, US

(21) Appl. No.: 11/726,678

(22) Filed: Mar. 22, 2007

Related U.S. Application Data
(60) Provisional application No. 60/785,400, filed on Mar. 22, 2006.

(51) Int. Cl. A61B 19/02

(52) U.S. Cl. 248/309.4; 206/350

(57) ABSTRACT

The apparatus includes a magnetic plate having a surface adapted to retain one or more surgical instruments. The magnetic plate is mounted for movable between various positions by a support arm such that the retained instruments are readily available for use as needed. A sterile cover is removably attached to the magnetic plate. The plate cover includes a sterile sheet. The sheet is situated proximate the surface of the magnetic plate. A handle is provided to manipulate the magnetic plate. A sterile cover is detachably mounted over the handle. The support arm includes a telescoping member and rotatable joints.

### CROSS-REFERENCE TO RELATED APPLICATIONS

Priority is claimed on Provisional Patent Application No. 60/785,400, filed Mar. 22, 2006.

### STATEMENT REGARDING FEDERALLY SPONSORED RESEARCH OR DEVELOPMENT

Not Applicable

### REFERENCE TO A “SEQUENCE LISTING”, A TABLE, OR A COMPUTER PROGRAM LISTING APPENDIX SUBMITTED ON COMPACT DISC

Not Applicable

### BACKGROUND OF THE INVENTION

1. Field of the Invention

The present invention relates to surgical instruments and more particularly to apparatus for holding surgical instruments in the immediate proximity of the operating surgeon in a sterile manner.

2. Description of Prior Art Including Information Disclosed Under 37 CFR 1.97 and 1.98

During surgical procedures, surgeons require immediate access to surgical instruments. At the same time, it is necessary that the surgical instruments be maintained in a sterile manner. The present invention relates to apparatus capable of holding surgical instruments and associated equipment within the most immediate proximity of the operating surgeon in a sterile manner.

The purpose of the invention is not necessarily to hold all the instruments requisite of a particular operation, but instead to hold the instruments that the surgeon determines are necessary to expedite the surgical process. Those instruments are retained by the apparatus of the present invention such that they will not be lost on a horizontal table amidst many other instruments and the surgeon will not have to wait for an assistant to locate and hand over a particular instrument requested by the surgeon. Further, the assistant need not try to anticipate which instruments are considered by the surgeon to be immediately necessary during the surgical procedure.

### BRIEF SUMMARY OF THE INVENTION

The present invention includes a magnetic surface. The magnetic surface is attached to a moveable arm. The arm can be adjusted to any position the surgeon requires. The arm can be mounted so as to extend from a wall or from the ceiling. It also can be incorporated into surgical lighting system.

A unique feature of the invention is that a disposable, sterile sleeve unit is placed over the magnetic plate, preferably by the surgeon or an assistant after the surgeon or assistant has successfully scrubbed and has made themselves sterile. The sleeve unit can be changed before or during the surgical procedure in an aseptic manner by the surgeon or assistant, as required.

Sleeve units for this purpose will be fabricated specifically for use in conjunction with the apparatus of the present invention. An aseptically prepared plastic attaching structure is provided to guide the sleeve unit and secure it in place on the magnetic plate. Suspended from the attaching structure is a standard and suitable surgical fabric. This fabric will be durable enough to resist wear from the surgical instruments while allowing the magnetic force of the plate to securely retain the surgical instruments attached to its surface.

Further, a disposable sterile handle cover is provided. The handle cover is placed over the handle to ensure sterility.

It is, therefore, a prime object of the present invention to provide apparatus for holding surgical instruments in a sterile manner.

It is another object of the present invention to provide apparatus for holding surgical instruments in a sterile manner utilizing a magnetic plate with a detachable sterile cover.

It is another object of the present invention to provide apparatus for holding surgical instruments in a sterile manner which is supported by a support arm that is movable to adjust the position and orientation of the magnetic plate.

It is another object of the present invention to provide apparatus for holding surgical instruments in a sterile manner which includes a handle for manipulating the magnetic plate and a sterile detachable cover for the handle.

In general, those objectives are achieved by the apparatus of the present invention as explained in detail in the following specification.

In accordance with the present invention, apparatus is provided for holding surgical instruments or the like. The apparatus includes a magnetic plate having a surface adapted to retain one or more surgical instruments. Means are provided for securing the magnetic plate such that it is movable between various positions such that the retained instruments are readily available for use as needed. The apparatus also includes sterile plate cover means and means for attaching the sterile plate cover means proximate the surface of the magnetic plate.

The apparatus includes a base from which the magnetic plate is suspended. The attaching means includes means for engaging the base.

The magnetic plate has first and second magnetic surfaces adapted to retain one or more surgical instruments. The sterile plate cover means includes first and second sterile sheets defining a recess therebetween. The magnetic plate is received within the recess between the sterile sheets.

The apparatus also includes a handle extending from the base. A sterile handle cover is provided. The handle cover is removably mounted on the handle. Means are provided for detachably mounting the handle cover on the handle.

The magnetic plate securing means takes the form of a support arm. The support arm includes a telescoping member. The support arm also includes means for rotating the magnetic plate.

### BRIEF DESCRIPTION OF THE SEVERAL VIEWS OF DRAWINGS

To these and to such other objects that may hereinafter appears, the present invention relates to apparatus for holding surgical instruments as described in detail in the following specification and recited in the annexed claims, taken together with the accompanying drawings, in which like numerals refer to like parts and in which:

FIG. 1 is a perspective view of the magnetic plate and the lower portion of the telescoping member support arm of the invention, showing the sterile plate cover exploded from the magnetic plate;

FIG. 2 is a perspective view of the magnetic plate with the cover mounted thereon and a scalpel retained on the magnetic plate, showing the telescoping support arm member extended;

FIG. 3 is a line drawing illustrating the various movements of a ceiling mounted embodiment of the support arm;

FIG. 4 is a front view of the magnetic plate without the cover;

FIG. 5 is a cross-sectional view of the handle;

FIG. 6 is a cross-sectional view of the handle cover; and

FIG. 7 is a cross-sectional view of the handle with the cover mounted thereon.

### DETAILED DESCRIPTION OF THE INVENTION

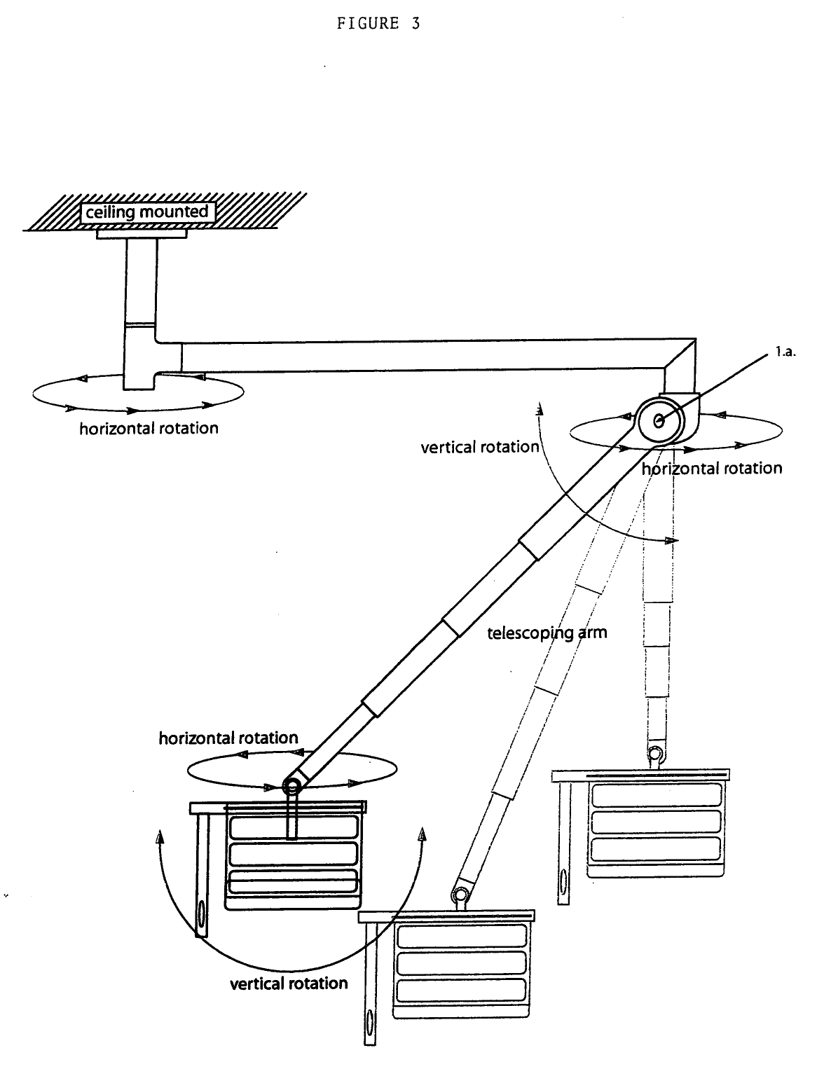

As illustrated in the drawings, numeral 1 refers generally to a telescoping and rotatable support arm that allows for the precise placement of the instrument holding portion of the apparatus of the present invention over an operating table (not shown). One end of support arm 1 is adapted to be secured to a wall, the ceiling or to a light fixture. The other end of support arm 1 is connected to the instrument holding portion of the apparatus by a rotational system which includes a joint 2, that allows for vertical rotation of the instrument holding portion and a joint 3, that allows for horizontal rotation of the instrument holding portion, as is illustrated in FIG. 3.

The instrument holding portion of the invention includes a base 4 from which the magnetic plate 6 is suspended. Base 4 houses the mechanism allowing for control of horizontal and vertical rotation of the magnetic plate.

The exterior surface on each side of base 4 is provided with an elongated groove. The grooves are adapted to receive inwardly directed elongated protrusions situated on the interior surfaces of the attaching means 11 of the sterile cover. The attaching means 11 has a generally “U” shaped cross-sectional shape. It is made of flexible material, such as plastic, configured to “snap-fit” over base 4, such that the sterile cover can be detachably mounted on the instrument holding portion.

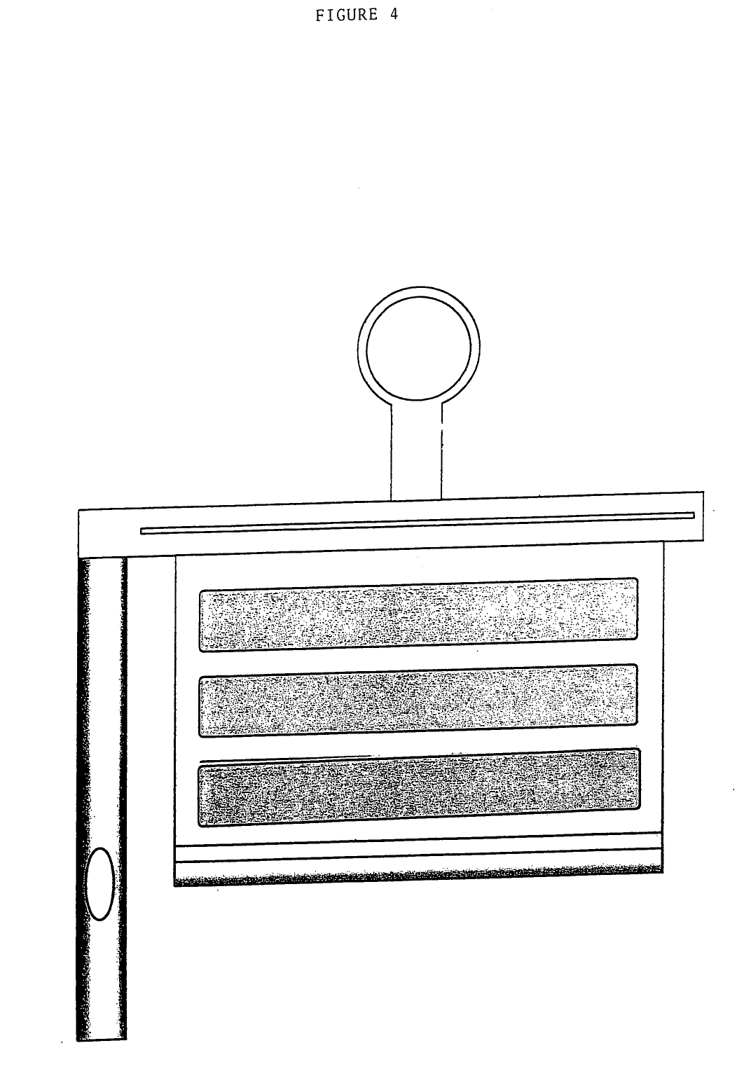

As best seen in FIG. 4, the side surfaces of magnetic plate 6 each have three recessed sections. Those recesses are hollowed out to a specific depth so as to reduce the contact area between the surface of the magnetic plate and the retained instrument. Limiting the contact area between the instrument and the surface of the magnetic plate ensures that the magnetic attraction between the plate and instrument will not be strong enough to prevent or interfere with the smooth retrieval of a retained instrument.

With this surface configuration, a maximum of four magnetic surface portions (proximate the top and bottom of the plate surface, as well as between the recesses) are available, each approximately ½ of an inch wide, allowing the placement of instruments of various weights on the magnetic surface. Further, this surface configuration also creates clearance between the plate surface and the instrument to allow the fingers of the surgeon to more securely grasp the instrument.

Located along the bottom edge of plate 6 is a plastic end 7 with a half radius bottom. End 7 reduces possible damage to an instrument if it is not properly placed on the plate. The end also provides a non-magnetic surface off of which to pivot the instrument upon its removal.

A handle 9, extending downwardly from a portion of base 4 that extends outwardly beyond the edge of magnetic plate 6, is provided such that the surgeon can maneuver the instrument holding portion into the desired position and orientation. The lower portion of handle 9 has a depressible button 10 which controls the rotatable joints 2, 3 of the support arm.

When button 10 is depressed, the rotatable joints 2, 3 below the telescoping member will rotate freely, such that the instrument holding portion can rotate in both the horizontal and vertical directions. When button 10 is not depressed, the rotatable joints 2, 3 below the telescoping member are locked such that the instrument holding portion will not rotate in either the horizontal or vertical direction. However, the telescoping member of the support arm will still be extendable and the rotational points above the telescoping member will still operate.

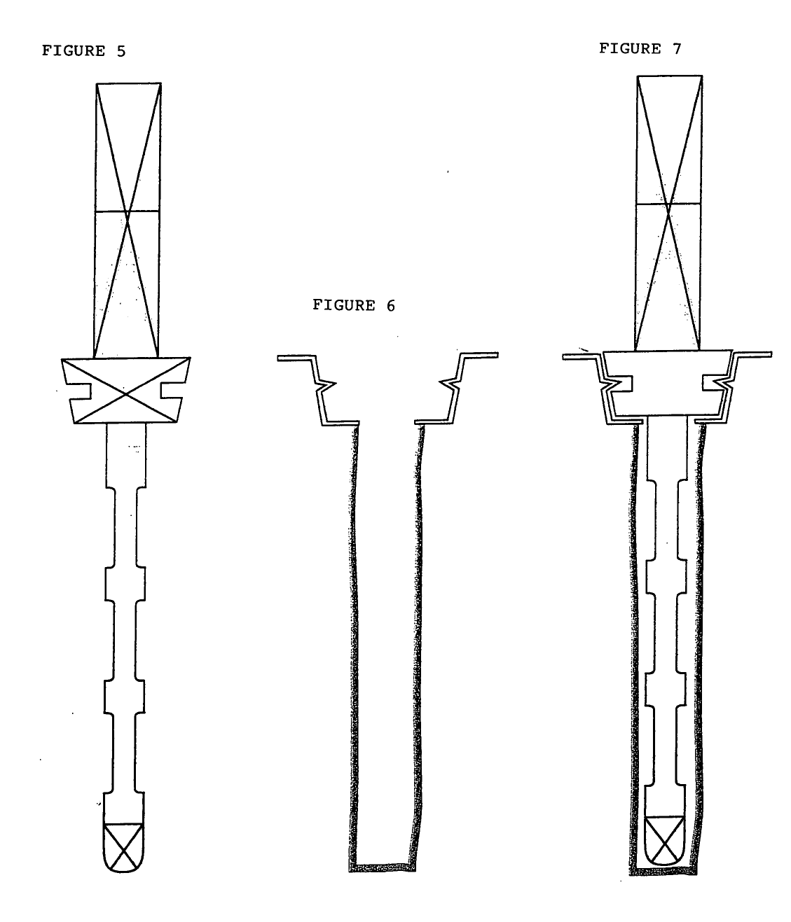

A sterile handle guard or cover 10 allows the surgeon to grasp the handle to manipulate the instrument holding portion without contaminating his or her sterile gloves. FIGS. 5, 6 and 7 illustrate the configuration of the handle, the shape of the handle cover and the structure that permits the handle cover to be detachably mounted over the handle.

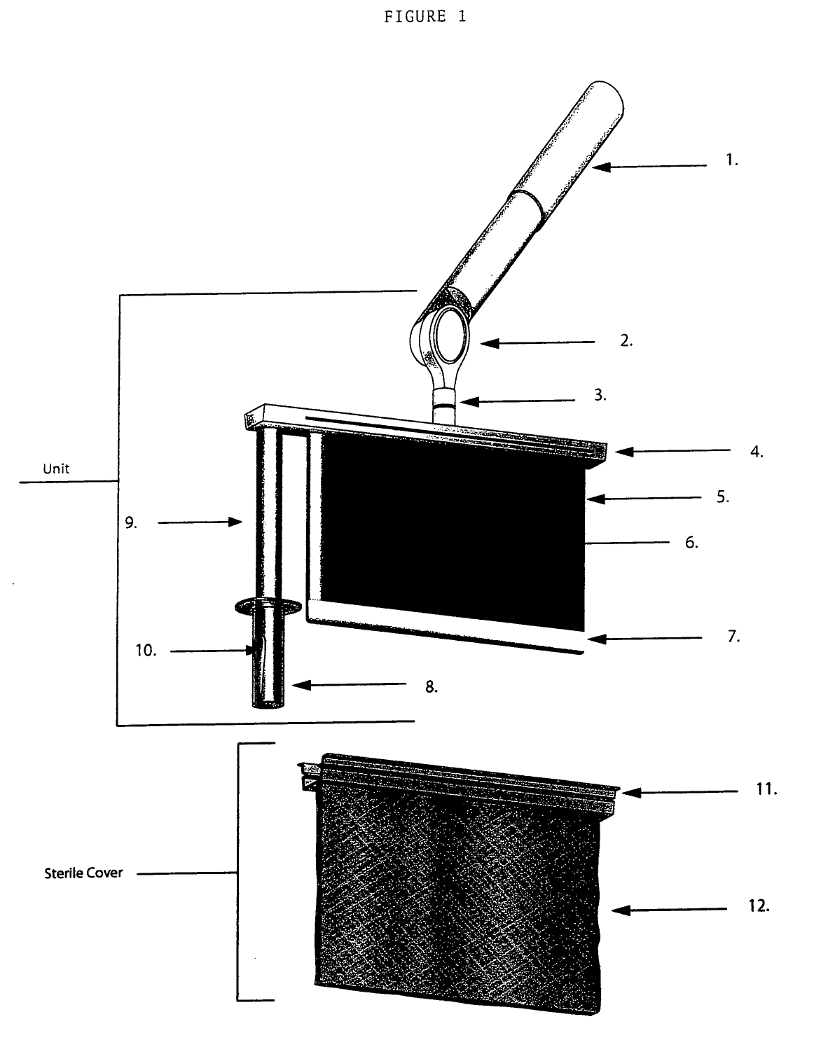

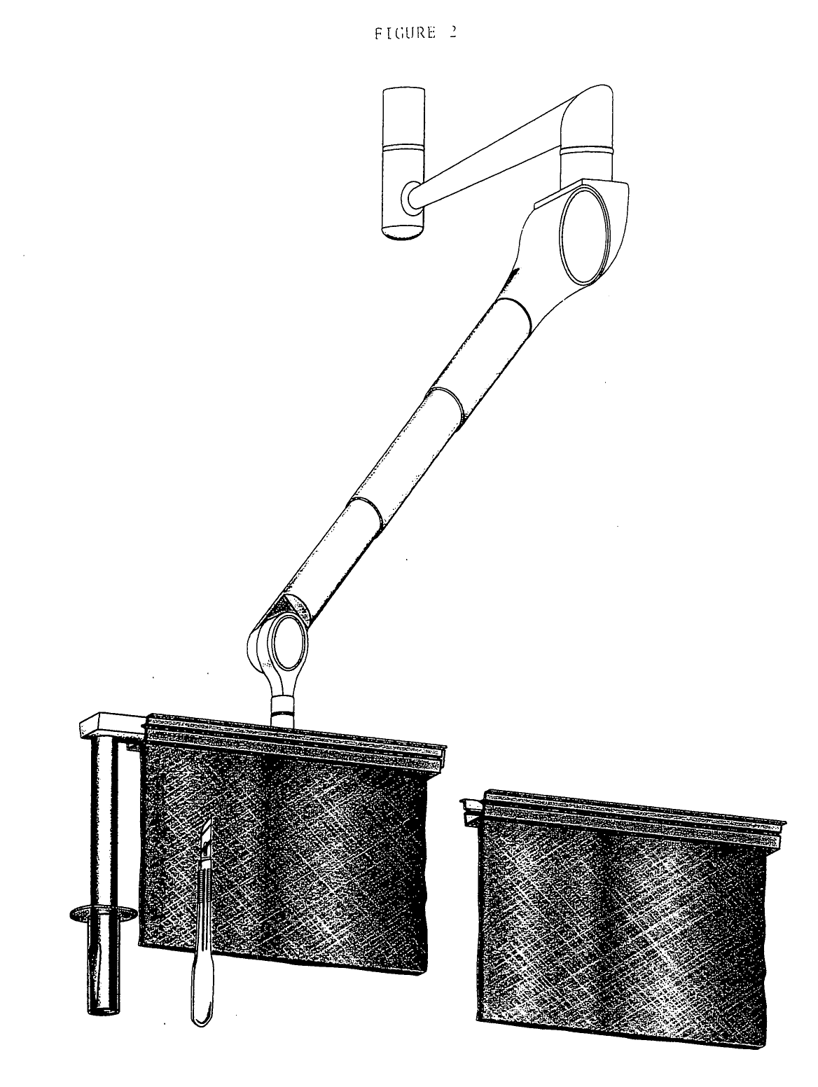

Referring again to FIG. 1, the sterile cover for the magnetic plate includes sterile surgical cloth or fabric means 12 suspended from attaching means 11. Although means 12 may have a single fabric sheet, preferably, means 12 takes the form of two side-by-side fabric sheets that define a recess therebetween. The magnetic plate is received within the recess in the sterile cover when the cover is mounted on the instrument holding portion by the engagement of attaching means 11 on base 4. Attaching means 11 is provided with an elongated slot or opening such that the magnetic plate can extend though the attaching means when the cover is mounted on the instrument holding portion. Accordingly, when the cover is mounted on the instrument holding portion, each sheet of means 12 is situated proximate a different magnetic surface of the magnetic plate.

As seen in FIG. 3, a control knob 1a is provided in the rotational joint immediately above the telescoping member of the support arm. The purpose of that knob is to adjust the amount of force needed for horizontal rotation of support arm.

While only a single preferred embodiment of the present invention has been disclosed for purposes of illustration, it is obvious that many modifications and variations could be made thereto. It is intended to cover all of those modifications and variations which fall within the scope of the present invention, as defined by the following claims.

I claim:

1. Apparatus for holding surgical instruments or the like comprising a magnetic plate having a surface adapted to retain one or more surgical instruments, means for securing said magnetic plate such that it is movable between various positions, sterile plate cover means and means for attaching said cover means proximate said surface of said magnetic plate.

2. The apparatus of claim 1, further comprising a base from which said magnetic plate is suspended and wherein said attaching means comprises means for engaging said base.

3. The apparatus of claim 1 wherein said magnetic plate has first and second magnetic surfaces and wherein said sterile plate cover means comprises first and second sterile sheets defining a recess therebetween adapted to receive said magnetic plate.

4. The apparatus of claim 2 further comprising a handle extending from said base.

5. The apparatus of claim 4 further comprising a sterile handle cover.

6. The apparatus of claim 5 wherein said handle cover is removably mounted on said handle.

7. The apparatus of claim 5 further comprising means for detachably mounting said handle cover on said handle.

8. The apparatus of claim 1 wherein said securing means comprises a support arm.

9. The apparatus of claim 8 wherein said support arm comprises a telescoping member.

10. The apparatus of claim 8 wherein said support arm comprises means for rotating said magnetic plate.
